SEQUENCE LISTING (22 sequences)

SEQ ID NO: 1 - protein, 39 aa, Heloderma suspectum
  SITE 39: note = Amidated residue
HGEGTFTSDLSKQMEEEAVRLFIEWLKNGGPSSGAPPPS

SEQ ID NO: 2 - protein, 39 aa, Heloderma horridum
  SITE 39: note = Amidated residue
HSDGTFTSDLSKQMEEEAVRLFIEWLKNGGPSSGAPPPS

SEQ ID NO: 3 - protein, 39 aa, synthetic construct
  SITE 39: note = Amidated residue
HGEGTFTSDLSKQLEEEAVRLFIEWLKNGGPSSGAPPPS

SEQ ID NO: 4 - protein, 39 aa, synthetic construct
  SITE 39: note = Amidated residue
HGEGTFTSDLSKQLEEEAVRLFIEFLKNGGPSSGAPPPS

SEQ ID NO: 5 - protein, 39 aa, synthetic construct
  SITE 39: note = Amidated residue
HGEGTFTSDLSKQLEEEAARLFIEFLKNGGPSSGAPPPS

SEQ ID NO: 6 - protein, 30 aa, synthetic construct
HGEGTFTSDLSKQMEEEAVRLFIEWLKNGG

SEQ ID NO: 7 - protein, 30 aa, synthetic construct
HGEGTFTSDLSKQLEEEAVRLFIEWLKNGG

SEQ ID NO: 8 - protein, 30 aa, synthetic construct
HGEGTFTSDLSKQLEEEAVRLFIEFLKNGG

SEQ ID NO: 9 - protein, 30 aa, synthetic construct
HGEGTFTSDLSKQLEEEAARLFIEFLKNGG

SEQ ID NO: 10 - protein, 28 aa, synthetic construct
HGEGTFTSDLSKQMEEEAVRLFIEWLKN

SEQ ID NO: 11 - protein, 28 aa, synthetic construct
HGEGTFTSDLSKQLEEEAVRLFIEWLKN

SEQ ID NO: 12 - protein, 28 aa, synthetic construct
HGEGTFTSDLSKQLEEEAVRLFIEFLKN

SEQ ID NO: 13 - protein, 28 aa, synthetic construct
HGEGTFTSDLSKQLEEEAARLFIEFLKN

SEQ ID NO: 14 - protein, 39 aa, synthetic construct
  SITE 39: note = Amidated residue
HGEGTFTSDLSKQLEEKAAKEFIEFLKQGGPSSGAPPPS

SEQ ID NO: 15 - protein, 39 aa, synthetic construct
  SITE 39: note = Amidated residue
HGEGTFTSDLSKQLEEKAAKEFIEWLKQGGPSSGAPPPS

SEQ ID NO: 16 - protein, 46 aa, synthetic construct
  SITE 39: note = Amidated residue
HGEGGGGGGGGTFTSDLSKQMEEEAVRLFIEWLKQGGPSSGAPPPS

SEQ ID NO: 17 - protein, 46 aa, synthetic construct
  SITE 39: note = Amidated residue
HGEGTFTSDLSKQLEEEAVRLFIEWLKQGGPSSGGGGGGGGAPPPS

SEQ ID NO: 18 - protein, 37 aa, synthetic construct
HGEFTFTSDLSKQLEEEAVRLFIEWLKQGGPSKEIIS

SEQ ID NO: 19 - protein, 39 aa, synthetic construct
  SITE 39: note = Amidated residue
HGEFTFTSDLSKQLEEKAAKEFIEWLKQGGPSSGAPPPS

SEQ ID NO: 20 - protein, 39 aa, synthetic construct
  SITE 39: note = Amidated residue
HGEGTFTSDLVKILEAEAVRKFIEFLKNGGPSSGAPPPS

SEQ ID NO: 21 - protein, 40 aa, synthetic construct
HGEGTFTSDLSKQMEEEAVRLFIEWLKNGGPSSGAPPPSK

SEQ ID NO: 22 - protein, 31 aa, unidentified
HAEGTFTSDVSSYLEGQAAKEFIAWLVKGRG

(19) United States
(12) Patent Application Publication
(10) Pub. No.: US 2024/0002792 A1
(43) Pub. Date: Jan. 4, 2024

(54) REPAIR OF OVARIAN DAMAGE AND DAMPENING OF INFLAMMATORY MICROENVIRONMENT BY ADMINISTRATION OF MONOCYTIC-GRANULOCYTIC PROGENITORS WITH IMMUNE MODULATORY ACTIVITIES

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, NIceville, FL (US)

(73) Assignee: CREATIVE MEDICAL TECHNOLOGIES, INC., Phoenix, AZ (US)

(21) Appl. No.: 18/318,465

(22) Filed: May 16, 2023

Related U.S. Application Data
(60) Provisional application No. 63/343,846, filed on May 19, 2022.

(51) Int. Cl. C12N 5/078

(52) U.S. Cl. CPC C12N 5/0634; C12N 2502/1352; C12N 2501/50

(57) ABSTRACT

The invention provides means, methods and some compositions of matter useful to treat ovarian failure. In one embodiment progenitor cells possessing ability to differentiate along the monocytic and granulocyte linages are utilized as a source of cytokines for stimulation of ovarian repair/regeneration. Generation of said cells, such as classically termed myeloid derived suppressor cells is performed from sources including cord blood, bone marrow, mobilized peripheral blood and pluripotent stem cells. The invention further provides means of suppressing fibrosis and ongoing inflammation associated with ovarian dysfunction.

### CROSS-REFERENCE TO RELATED APPLICATIONS

This application claims priority to U.S. Provisional Application No. 63/343,846, titled “Repair of Ovarian Damage and Dampening of Inflammatory Microenvironment by Administration of Monocytic-Granulocytic Progenitors with Immune Modulatory Activities” filed May 19, 2022, which is hereby incorporated by reference herein in its entirety.

### SEQUENCE LISTING

The instant application contains a Sequence Listing which has been submitted electronically in XML format and is hereby incorporated by reference in its entirety. Said XML copy, created on Sep. 19, 2023, is named CMT_RODDIMA_NP1_SL.xml and is 24,836 bytes in size.

### FIELD OF THE INVENTION

The invention relates to the treatment and/or prevention of ovarian damage utilizing immune modulating cell populations.

### BACKGROUND OF THE INVENTION

Although regenerative medicine means for treating ovarian failure in animal models have been reported, little clinical translation of this work has occurred at a large scale. At a small scale, some reports have been published. In one study umbilical cord mesenchymal stem cells on a collagen scaffold (collagen/UC-MSCs) can activate primordial follicles in vitro via phosphorylation of FOXO3a and FOXO1. Transplantation of collagen/UC-MSCs to the ovaries of POF patients rescued overall ovarian function, evidenced by elevated estradiol concentrations, improved follicular development, and increased number of antral follicles. Successful clinical pregnancy was achieved in women with POF after transplantation of collagen/UC-MSCs or UC-MSCs. In summary, collagen/UC-MSC transplantation may provide an effective treatment for POF [1]. In another study, poor ovarian responders (PORs). POR women were divided into mesenchymal stroma cell (MSC) therapy (n=15) and routine ICSI (n=16) groups. The cultured Men-MSCs were autologously injected into left ovary of MSC group after approval by flow cytometry, karyotyping, endotoxin, sterility and mycoplasma tests. Changes in anti-Mullerian hormone (AMH), antral follicles count (AFC), oocytes and embryos number, clinical pregnancy rate and live birth rate were followed in both groups up to one year after treatment. 4 of 15 participants in MSC group got naturally pregnant during 3 months after cell administration, in contrast to no natural conception in control group (P=0.04). The mean AMH level did not significantly differ with that of previous cycle or control group. Although mean AFC and oocytes number in MSC group did not indicate considerable difference with those of control group, raise of these parameters in comparison with previous cycle was significant (both P=0.01). Nonetheless, oocyte fertilization rate and embryo number in MSC group were higher than control group (P=0.04 and P=0.008, respectively). Altogether, 7 of 15 women in MSC group and 2 of 16 women in routine ICSI group had clinical pregnancy that resulted in 5 live births in main group and one birth in control group. In conclusion, cell therapy using Men-MSCs could be considered as a potential treatment to restore fertility capability of POR women.

Igboeli et al. present two cases of Caucasian women with premature ovarian failure who resumed ovarian estrogen production and menses 7 months following autologous bone marrow-derived mesenchymal stem cell injections into the ovary. In this report, data is presented from the first two cases that have completed study procedures so far. The bone marrow-derived mesenchymal stem cells were harvested from the bone marrow of the iliac crest of the patients with premature ovarian failure and nucleated cells concentrated and enriched in bone marrow-derived mesenchymal stem cells intraoperatively, and then injected into the patient's right ovary via laparoscopy. Autologous bone marrow stem cell engraftment into the ovary resulted in several improvements in the treated patients with premature ovarian failure. In measurements by transvaginal ultrasound, there were increases of approximately 50% in volume of the treated ovaries in comparison with the contralateral control ovaries that persisted to the end of the study (1 year). Serum levels of estrogen increased by approximately 150% compared with the preoperative levels. Each of the two patients had an episode of menses, and also both of them reported marked improvement of their menopausal symptoms that also persisted to the end of the study (1 year). The bone marrow-derived mesenchymal stem cell implantation procedure was very well tolerated with no reported adverse events. The study reveals promising improvement of premature ovarian failure-related clinical manifestations in two patients after intraovarian autologous bone marrow-derived mesenchymal stem cells engraftment. These early observations call for additional assessment and further development of intraovarian bone marrow-derived mesenchymal stem cell injection for possible treatment of patients with premature ovarian failure [3]. In another study, 17 women who are poor responders to fertility approaches were treated by ovarian infusion of bone marrow-derived stem cells. Serum antimullerian hormone levels and antral follicular count (AFC), punctured follicles, and oocytes retrieved after stimulation (controlled ovarian stimulation) were measred. Apheresis was analyzed for growth factor concentrations. Treatment resulted in a significant improvement in AFC 2 weeks after treatment. With an increase in AFC of three or more follicles and/or two consecutive increases in antimullerian hormone levels as success criteria, ovarian function improved in 81.3% of women. These positive effects were associated with the presence of fibroblast growth factor-2 and thrombospondin. During controlled ovarian stimulation, autologous stem cell transplant increased the number of stimulable antral follicles and oocytes, but the embryo euploidy rate was low (16.1%). Five pregnancies were achieved: two after ET, three by natural conception [4]. A non-randomized clinical trial, phase I. Nine women with a definitive diagnosis of POF were divided into three groups (n=3 per group) that received either 5×10⁶, 10×10⁶, or 15×10⁶ autologous ADSCs suspension transplanted in the one ovary. Participants were followed-up at 24 h after the transplantation, and at 1 and 2 weeks, and 1, 2, 3, 6, and 12 months after the transplantation. The primary objective was to evaluate the safety of ADSCs transplantation. Secondary objectives included the effects of ADSCs transplantation on the resumption of menstruation, hormones level (Follicle-stimulating hormone (FSH) and anti-Mullerian hormone), ovarian function (Antral follicle count and ovary volume by ultrasonography evaluation) as well as dose escalation. Participants had not shown any early-onset possible side effects and secondary complications during follow-up. The menstruation resumption was observed in four patients which established for several months. In the 15×10⁶ group, two POF patients had a return of menstruation second months after the intervention. Two other POF patients in 5×10⁶ and 10×10⁶ cell groups reported menstruation resumption at 1 month after the intervention. We observed decreased serum FSH levels of less than 25 IU/1 in four patients. In two patients in 5×10⁶ and 10×10⁶ cell groups, serum FSH showed an inconsistent decline during a 1 year follow up after ADSCs transplantation. The ovarian volume, AMH, and AFC were variable during the follow-up and no significant differences between cell groups (p>0.05). The authors showed the intra-ovarian embedding of ADSCs is safe and feasible and is associated with an inconsistent decline in serum FSH. This should be further investigated with a large RCT [5]. In a larger study, 61 patients diagnosed with POI participated in this study. UCMSCs were isolated and cultured according to GMP standards, and then transplanted to the patients' ovary by orthotopic injection under the guidance of vaginal ultrasound. We monitored side effects, vital signs and changes in clinical and collected haematological and imaging parameters during the follow-ups. All patients showed normal clinical behavior without serious side effects or complications relevant to the treatment. Transplantation of UCMSCs rescued the ovarian function of POI patients, as indicated by increased follicular development and improved egg collection. POI patients who experienced shorter amenorrhoea durations (<1 year) seemed to obtain mature follicles more easily after stem cell therapy, and patients with better ovarian conditions (pre-operative antral follicles) were more likely to derive the better outcomes by UCMSC injection. Four successful clinical deliveries were obtained from POI patients after UCMSC transplantation, and all of these babies are developed normally [6]. Another large study involved woman with a diagnosis of POF and infertility. This multicenter study was performed at Jevremova Special Hospital in Belgrade, Saint James Hospital (Malta), and Remedica Skoplje Hospital, between 2015 and 2018. All patients went through numerous laboratory testings, including hormonal status. The autologous bone marrow mesenchymal stem cells (BMSCs) and growth factors were used in combination for activation of ovarian tissue before its re-transplantation. The software package SPSS 20.0 was used for statistical analysis of the results. Differences in follicle stimulating hormone (FSH), luteinizing hormone (LH), estradiol (E2), and progesterone (PG) hormone concentrations before and after 3, 6, and 12 months post-transplantation were tested in correlation with the volume of transplanted ovarian tissue. A significant correlation (P=0.029) was found between the change in E2 level after 3 months and the volume of re-transplanted tissues. Also after re-transplantation, 64% of the patients had follicles resulting in aspiration of oocytes in 25% of positive women with follicles. It was concluded that the SEGOVA method could potentially solve many human reproductive problems in the future due to the large number of patients diagnosed with POF, as well as the possibility of delaying menopause, thus improving the quality of life and general health [7].

As seen from the above, although there are some signals of potential efficacy, there have been no treatments that have produced widespread positive results for ovarian failure. In this current disclosure we present means of increasing regenerative activity in the ovary of patients with ovarian failure through administration of progenitor cells that are capable of differentiating into monocytic and granulocytic cells.

### SUMMARY

Preferred embodiments are directed to methods of preventing or treating ovarian failure comprising the steps of: a) identifying a patient suffering from ovarian failure or at risk of ovarian failure; b) withdrawing from said patient a population of myeloid lineage cells; c) contacting said myeloid lineage cells with a mesenchymal stem cell population and/or products generated from said mesenchymal stem cell population; d) optionally pulsing said myeloid cell population with one or more antigens associated with ovarian failure; e) extracting microvesicles from said myeloid cell population; and f) administering said microvesicles from said myeloid cell population into a patient in need of prophylaxis or treatment.

Preferred methods include embodiments wherein said risk of ovarian failure is quantified by one or more selected from the group consisting of: a) increase production of interferon gamma from T cells responding to a ovarian antigen as compared to T cells from an age-matched subject; b) decreased production of interleukin-4 from T cells responding to a ovarian antigen as compared to T cells from an age-matched subject; c) increased antibodies to a ovarian antigen as compared to T cells from an age-matched subject; d) increased fibrosis in the ovarian follicle; and e) decreased production of estrogen.

Preferred methods include embodiments wherein said T cells are selected from a group of T cells comprising of: a) CD3 T cells; b) CD4 T cells; c) CD8 T cells; d) Th1 T cells; e) Th2; f) Th3 T cells; g) Th9 T cells; h) Th17 T cells and i) Th22 T cells.

Preferred methods include embodiments wherein said antibody is a complement fixing antibody.

Preferred methods include embodiments wherein said antibody possesses the isotype IgG2b.

Preferred methods include embodiments wherein said myeloid cell population comprises monocytes.

Preferred methods include embodiments wherein said myeloid cell population comprises monocytic progenitors.

Preferred methods include embodiments wherein said myeloid cell population comprises macrophages.

Preferred methods include embodiments wherein said myeloid cell population comprises dendritic cells.

Preferred methods include embodiments wherein said myeloid cell population comprises dendritic cell progenitors.

Preferred methods include embodiments wherein said myeloid cell population comprises myeloid suppressor cells.

Preferred methods include embodiments wherein said myeloid cell population comprises myeloid suppressor cell progenitors.

Preferred methods include embodiments wherein said mesenchymal stem cells are derived from fluids.

Preferred methods include embodiments wherein said fluid is plasma.

Preferred methods include embodiments wherein said fluid is cerebral spinal fluid.

Preferred methods include embodiments wherein said fluid is urine.

Preferred methods include embodiments wherein said fluid is seminal fluid.

Preferred methods include embodiments wherein said mesenchymal stem cells are derived from tissues.

Preferred methods include embodiments wherein said tissue derived mesenchymal stem cells are selected from the group consisting of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Preferred methods include embodiments wherein said tissue derived mesenchymal stem cells are isolated from tissues containing cells selected from a group of cells comprising of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, and salivary gland mucous cells.

Preferred methods include embodiments wherein said mesenchymal stem cells are plastic adherent.

Preferred methods include embodiments wherein said mesenchymal stem cells express a marker selected from the group consisting of: a) CD73; b) CD90; and c) CD105.

Preferred methods include embodiments wherein said mesenchymal stem cells are derived from umbilical cord tissue and lack expression of a marker selected from the group consisting of: a) CD14; b) CD45; and c) CD34.

Preferred methods include embodiments wherein said mesenchymal stem cells from umbilical cord tissue express markers selected from the group consisting of; a) oxidized low density lipoprotein receptor 1, b) chemokine receptor ligand 3; and c) granulocyte chemotactic protein.

Preferred methods include embodiments wherein said mesenchymal stem cells from umbilical cord tissue do not express markers selected from the group consisting of: a) CD117; b) CD31; c) CD34; and CD45;

Preferred methods include embodiments wherein said mesenchymal stem cells from umbilical cord tissue express, relative to a human fibroblast, increased levels of interleukin 8 and reticulon 1

Preferred methods include embodiments wherein said mesenchymal stem cells from umbilical cord tissue have the potential to differentiate into cells of at least a skeletal muscle, vascular smooth muscle, pericyte or vascular endothelium phenotype.

Preferred methods include embodiments wherein said mesenchymal stem cells from umbilical cord tissue express markers selected from the group consisting of: a) CD10; b) CD13; c) CD44; d) CD73; and e) CD90.

Preferred methods include embodiments wherein said umbilical cord tissue mesenchymal stem cell is an isolated umbilical cord tissue cell isolated from umbilical cord tissue substantially free of blood that is capable of self-renewal and expansion in culture,

Preferred methods include embodiments wherein said umbilical cord tissue mesenchymal stem cells has the potential to differentiate into cells of other phenotypes.

Preferred methods include embodiments wherein said other phenotypes comprise: a) osteocytic; b) adipogenic; and c) chondrogenic differentiation.

Preferred methods include embodiments wherein said cord tissue derived mesenchymal stem cells can undergo at least 20 doublings in culture.

Preferred methods include embodiments wherein said cord tissue derived mesenchymal stem cell maintains a normal karyotype upon passaging

Preferred methods include embodiments wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

Preferred methods include embodiments wherein said cord tissue mesenchymal stem cells does not express one or more markers selected from the group consisting of; a) CD31; b) CD34; c) CD45; d) CD80; e) CD86; f) CD117; g) CD141; h) CD178; i) B7-H2; j) HLA-G and k) HLA-DR,DP,DQ.

Preferred methods include embodiments wherein said umbilical cord tissue-derived cell secretes factors selected from the group consisting of: a) MCP-1; b) MIP1beta; c) IL-6; d) IL-8; e) GCP-2; f) HGF; g) KGF; h) FGF; i) HB-EGF; j) BDNF; k) TPO; 1) RANTES; and m) TIMP1

Preferred methods include embodiments wherein said umbilical cord tissue derived cells express markers selected from the group consisting of: a) TRA1-60; b) TRA1-81; c) SSEA3; d) SSEA4; and e) NANOG.

Preferred methods include embodiments wherein said umbilical cord tissue-derived cells are positive for alkaline phosphatase staining.

Preferred methods include embodiments wherein said microvesicles are exosomes.

Preferred methods include embodiments wherein said microvescicles are apoptotic bodies.

Preferred methods include embodiments wherein said progenitor cells of the myeloid and granulocytic lineages are administered directly into the ovary.

### DETAILED DESCRIPTION OF THE INVENTION

The invention provides means of preventing and/or reversing ovarian failure through administration of myeloid cells or microvesicles derived from myeloid cells that have been programmed with regenerative cells such as mesenchymal stem cells. Reprogramming includes direct contact and/or culture with conditioned media of myeloid derived cells from the patient with said regenerative cells. The administration of cells that have been treated with enhancing factors or conditions is disclosed under the current invention as means of stimulating the generation of ovarian tissue, as well as increase production of various cytokines and hormones. In some embodiments the invention teaches increase production of ovarian hormones by treatment with myeloid cells. In some embodiments the invention teaches the use of myeloid derived suppressor cells for stimulation of ovarian regeneration.

In one embodiment of the invention, regeneration of ovarian tissue is induced by administration of autologous myeloid derived suppressor cells that have been collected from mobilization of peripheral blood cells by treatment with a cytokine such as G-CSF. These mobilized cells are then purified for expression of myeloid markers such as CD34, CD33, CD133, GR-1, and flk-1, concentrated, and administered into the ovary.

In other embodiments, peripheral blood mononuclear cells that have been cultured with regenerative cells. In one embodiment said regenerative cells are umbilical cord mesenchymal stem cells. In one embodiment cells are cultured at a ratio of 1 peripheral blood mononuclear cell to one umbilical cord mesenchymal stem cell. In one embodiment cells are

Throughout this specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the disclosure may “consist essentially of” or “consist of” one or more sequences of the invention, for example. Some embodiments may consist of or consist essentially of one or more elements, method steps, and/or methods of the invention. It is contemplated that any method or composition described herein can be implemented with respect to any other method or composition described herein. The scope of the present application is not intended to be limited to the particular embodiments of the process, machine, manufacture, composition of matter, means, methods and steps described in the specification.

Throughout this specification, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe, etc. A second exemplary method of administering is by a direct mechanism such as, local tissue administration, oral ingestion, transdermal patch, topical, inhalation, suppository, etc.

The term “allogeneic,” as used herein, refers to cells of the same species that differ genetically from cells of a host.

The term “autologous,” as used herein, refers to cells derived from the same subject. The term “engraft” as used herein refers to the process of stem cell incorporation into a tissue of interest in vivo through contact with existing cells of the tissue.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

As used herein, the term “activated mesesnchymal stem cell” refers to mesenchymal stem cells treated with one or more agents and/or stimuli capable of inducing one or more alterations in the cell: metabolic, immunological, growth factor-secreting, surface marker expression, and/or production of microvesicles. Examples of agents include epidermal growth factor (EGF; (Peprotech), Transforming Growth Factor-alpha (TGF-alpha; Peprotech), basic Fibroblast Growth Factor (bFGF; Peprotech), brain-derived neurotrophic factor (BDNF; R&D Systems), and Keratinocyte Growth Factor (KGF; Peprotech). EGF is a potent mitogenic factor for a variety of cultured ectodermal and mesodermal cells and has a profound effect on the differentiation of specific cells in vivo and in vitro and of some fibroblasts in cell culture. The EGF precursor exists as a membrane-bound molecule which is proteolytically cleaved to generate the 53-amino acid peptide hormone that stimulates cells. A preferred mitogenic growth factor is EGF. EGF is preferably added to the basal culture medium at a concentration of between 5 and 500 ng/ml or of at least 5 and not higher than 500 ng/ml. A preferred concentration is at least 10, 20, 25, 30, 40, 45, or 50 ng/ml and not higher than 500, 450, 400, 350, 300, 250, 200, 150, or 100 ng/ml. A more preferred concentration is at least 50 and not higher than 100 ng/ml. An even more preferred concentration is about 50 ng/ml or 50 ng/ml. The same concentrations could be used for a FGF, preferably for FGF10 or FGF7. If more than one FGF is used, for example, FGF7 and FGF10, the concentration of a FGF is as defined above and refers to the total concentration of FGF used. During culturing of stem cells, the mitogenic growth factor is preferably added to the culture medium every second day, while the culture medium is refreshed preferably every fourth day. Any member of the bFGF family may be used. In some cases, FGF7 and/or FGF10 is used. FGF7 is also known as KGF (Keratinocyte Growth Factor).

“Cell culture” is an artificial in vitro system containing viable cells, whether quiescent, senescent or (actively) dividing. In a cell culture, cells are grown and maintained at an appropriate temperature, typically a temperature of 37.degree. C. and under an atmosphere typically containing oxygen and CO.sub.2. Culture conditions may vary widely for each cell type though, and variation of conditions for a particular cell type can result in different phenotypes being expressed. The most commonly varied factor in culture systems is the growth medium. Growth media can vary in concentration of one or more of nutrients, growth factors, and the presence of other components. The growth factors used to supplement media are often derived from animal blood, such as calf serum.

As used herein, the term “conditioned medium of regenerative cells” refers to a liquid media that has been in contact with cells, wherein the cells produce one or more factors that enter the media, thus bestowing upon the media at least one therapeutic activity.

The term “individual”, as used herein, refers to a human or animal that may or may not be housed in a medical facility and may be treated as an outpatient of a medical facility. The individual may or may not be receiving one or more medical compositions from a medical practitioner and/or via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method and/or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

As used herein, “subject” includes but is not limited to human, non-human primates (e.g., monkey, ape), sheep, rabbit, pig, dog, cat, mouse, or rat.

As used herein, “transplant rejection” is defined as the nearly complete, or complete, loss of viable graft tissue from the recipient subject.

As used herein, “ligand” refers to a molecule that specifically recognizes and binds another molecule, for example, a ligand for CTLA4 is a CD80 and/or CD86 molecule.

As used herein, “a soluble ligand which recognizes and binds CD80 and/or CD86 antigen” includes ligands such as CTLA4Ig, CD28Ig or other soluble forms of CTLA4 and CD28; recombinant CTLA4 and CD28; mutant CTLA4 molecules such as L104EA29YIg; and any antibody molecule, fragment thereof or recombinant binding protein that recognizes and binds a CD80 and/or CD86 antigen. These agents are also considered “immunosuppressive agents”.

As used herein, “costimulatory pathway” is defined as a biochemical pathway resulting from interaction of costimulatory signals on T cells and antigen presenting cells (APCs). Costimulatory signals help determine the magnitude of an immunological response to an antigen. One costimulatory signal is provided by the interaction with T cell receptors CD28 and CTLA4 with CD80 and/or CD86 molecules on APCs.

As used herein, “CD80 and/or CD86” includes B7-1 (also called CD80). B7-2 (also called CD86), B7-3 (also called CD74), and the B7 family, e.g., a combination of B7-1, B7-2, and/or B7-3.

As used herein, “costimulatory blockade” is defined as a protocol of administering to a subject, one or more agents that interfere or block a costimulatory pathway, as described above. Examples of agents that interfere with the costimulatory blockade include, but are not limited to, soluble CTLA4, mutant CTLA4, soluble CD28, anti-B7 monoclonal antibodies (mAbs), soluble CD40, and anti-gp39 mAbs. In one embodiment, L104EA29YIg is a preferred agent that interferes with the costimulatory blockade.

As used herein, “T cell depleted bone marrow” is defined as bone marrow removed from bone that has been exposed to an anti-T cell protocol. An anti-T cell protocol is defined as a procedure for removing T cells from bone marrow. Methods of selectively removing T cells are well known in the art. An example of an anti-T cell protocol is exposing bone marrow to T cell specific antibodies, such as anti-CD3, anti-CD4, anti-CD5, anti-CD8, and anti-CD90 monoclonal antibodies, wherein the antibodies are cytotoxic to the T cells. Alternatively, the antibodies can be coupled to magnetic particles to permit removal of T cells from bone marrow using magnetic fields. Another example of an anti-T cell protocol is exposing bone marrow T cells to anti-lymphocyte serum or anti-thymocyte globulin.

As used herein, “tolerizing dose of T cell depleted bone marrow” is defined as an initial dose of T cell depleted bone marrow that is administered to a subject for the purpose of inactivating potential donor reactive T cells.

As used herein, “engrafting dose of T cell depleted bone marrow” is defined as a subsequent dose of T cell depleted bone marrow that is administered to a subject for the purpose of establishing mixed hematopoietic chimerism. The engrafting dose of T cell depleted bone marrow will accordingly be administered after the tolerizing dose of T cell depleted bone marrow. In one embodiment the invention discloses use of hematopoietic transplantation to treat ovarian failure.

As used herein, “mixed hematopoietic chimerism” is defined as the presence of donor and recipient blood progenitor and mature cells (e.g., blood deriving cells) in the absence (or undetectable presence) of an immune response.

As used herein, “administer” or “administering” to a subject includes but not limited to intravenous (i.v.) administration, intraperitoneal (i.p.) administration, intramuscular (i.m.) administration, subcutaneous administration, oral administration, administration by injection, as a suppository, or the implantation of a slow-release device such as a miniosmotic pump, to the subject.

As used herein, the term “exendin” includes naturally occurring (or synthetic versions of naturally occurring) exendin peptides that are found in the salivary secretions of the Gila monster. Exendins of particular interest include exendin-3 and exendin-4. The exendins, exendin analogs, and exendin agonists for use in the methods described herein may optionally be amidated, and may also be in an acid form, pharmaceutically acceptable salt form, or any other physiologically active form of the molecule. Exendin-4 (HGEGTFTSDLSKQMEEEAVRLFIEWLKNGGPSSGAPPPS-NH.sub.2 (SEQ ID NO:1)) is a peptide found in the saliva of the Gila monster, Heloderma suspectum; and exendin-3 (HSDGTFTSDLSKQMEEEAVRLFIEWLKNGG PSSGAPPPS-NH.sub.2 (SEQ ID NO:2)) is a peptide found in the saliva of the beaded lizard, Heloderma horridum. Exendins have some amino acid sequence similarity to some members of the glucagon-like peptide (GLP) family. For example, exendin-4 has about 53% sequence identity with glucagon-like peptide-1(GLP-1)(7-37) (HAEGTFTSDVSSYLEGQAAKEFIAWLVKGRG (SEQ ID NO: 22). However, exendin-4 is transcribed from a distinct gene, not the Gila monster homolog of the mammalian proglucagon gene from which GLP-1 is expressed. Additionally, exendin-4 is not an analog of GLP-1(7-37) because the structure of synthetic exendin-4 peptide was not created by sequential modification of the structure of GLP-1. Nielsen et al, Current Opinion in Investigational Drugs, 4(4):401-405 (2003). Synthetic exendin-4, also known as exenatide, is commercially available as BYETTA® (Amylin Pharmaceuticals, Inc. and Eli Lilly and Company). BYETTA® contains exenatide, a preservative (e.g., metacresol), a tonicity-adjusting agent (e.g., mannitol), and a buffer (e.g., an acetate buffer). A once weekly formulation of exenatide is currently awaiting FDA approval and is described in WO 2005/102293, the disclosure of which is incorporated by reference herein. This once weekly formulation comprises exenatide and biodegradable polymeric (e.g., poly(lactide-co-glycolide)) microspheres, and is referred to herein as EQW (BYDUREON™ by Amylin Pharmaceuticals, Inc., Eli Lilly and Company, Alkermes, Inc.). In one embodiment of the invention exendin or exendin analogs are utilized as adjuvants with myeloid derived suppressor cells to repaired damaged ovaries.

As used herein, “Exendin analog” refers to peptides or other compounds which elicit a biological activity of an exendin reference peptide, preferably having a potency equal to or better than the exendin reference peptide (e.g., exendin-4), or within five orders of magnitude (plus or minus) of potency compared to the exendin reference peptide, when evaluated by art-known measures such as receptor binding and/or competition studies as described, e.g., by Hargrove et al, Regulatory Peptides, 141:113-119 (2007), the disclosure of which is incorporated by reference herein. Preferably, the exendin analogs will bind in such assays with an affinity of less than 1 uM, and more preferably with an affinity of less than 3 nM, or less than 1 nM. The term “exendin analog” may also be referred to as “exendin agonist”. Exendin analogs also include the peptides described herein which have been chemically derivatized or altered, for example, peptides with non-natural amino acid residues (e.g., taurine, .beta.-amino acid residues, .gamma.-amino acid residues, and D-amino acid residues), C-terminal functional group modifications, such as amides, esters, and C-terminal ketone modifications and N-terminal functional group modifications, such as acylated amines, Schiff bases, or cyclization, as found, for example, in the amino acid pyroglutamic acid. Exendin analogs may also contain other chemical moieties, such as peptide mimetics. Exemplary exendins and exendin analogs include exendin-4 (SEQ ID NO:1); exendin-3 (SEQ ID NO:2); Leu.sup.14-exendin-4 (SEQ ID NO:3); Leu.sup.14,Phe.sup.25-exendin-4 (SEQ ID NO:4); Leu.sup.14,Ala.sup.19,Phe.sup.25-exendin-4 (SEQ ID NO:5); exendin-4(1-30) (SEQ ID NO:6); Leu.sup.14-exendin-4(1-30) (SEQ ID NO:7); Leu.sup.14,Phe.sup.25-exendin-4(1-30) (SEQ ID NO:8); Leu.sup.14,Ala.sup.19,Phe.sup.25-exendin-4(1-30) (SEQ ID NO:9); exendin-4(1-28) (SEQ ID NO:10); Leu.sup.14-exendin-4(1-28) (SEQ ID NO:11); Leu.sup.14,Phe.sup.25-exendin-4(1-28) (SEQ ID NO:12); Leu.sup.14,Ala.sup.19,Phe.sup.25-exendin-4 (1-28) (SEQ ID NO:13); Leu.sup.14,Lys.sup.17,20,Ala.sup.19,Glu.sup.21,Phe.sup.25,Gln.sup.28-exen-din-4 (SEQ ID NO:14); Leu.sup.14,Lys.sup.17,20,Ala.sup.19,Glu.sup.21,Gln.sup.28-exendin-4 (SEQ ID NO:15); octylGlyl.sup.4,Gln.sup.28-exendin-4 (SEQ ID NO:16); Leu.sup.14,Gln.sup.28,octylGly.sup.34-exendin-4 (SEQ ID NO:17); Phe.sup.4,Leu.sup.14,Gln.sup.28,Lys.sup.33,Glu.sup.34, Ile.sup.35,36,Ser.sup.37-exendin-4(1-37) (SEQ ID NO:18); Phe.sup.4,Leu.sup.14,Lys.sup.17,20,Ala.sup.19,Glu.sup.21,Gln.sup.28-exend-in-4 (SEQ ID NO:19); Val.sup.11,Ile.sup.13,Leu.sup.14,Ala.sup.16,Lys.sup.21,Phe.sup.25-exendin-4 (SEQ ID NO:20); exendin-4-Lys.sup.40 (SEQ ID NO:21); lixisenatide (Sanofi-Aventis/Zealand Pharma); CJC-1134 (ConjuChem, Inc.); [N.sup..epsilon.-(17-carboxyheptadecanoic acid)Lys.sup.20]exendin-4-NH.sub.2; [N.sup..epsilon.-(17-carboxyhepta-decanoyl)Lys.sup.32]exendin-4-NH.sub.2; [desamino-His.sup.1,N.sup..epsilon.-(17-carboxyheptadecanoyl)Lys.sup.20]e-xendin-4-NH.sub.2; [Arg.sup.12,27,NLe.sup.14,N.sup.E-(17-carboxy-heptadecanoyl)Lys.sup.32]ex-endin-4-NH.sub.2; [N.sup..epsilon.-(19-carboxy-nonadecanoylamino)Lys.sup.20]-exendin-4-NH.s-ub.2; [N-(15-carboxypentadecanoylamino)Lys.sup.20]-exendin-4-NH.sub.2; [N.sup.E-(13-carboxytridecanoylamino)Lys.sup.20]exendin-4-NH.sub.2; [N.sup..epsilon.-(11-carboxy-undecanoyl-amino)Lys.sup.20]exendin-4-NH.sub-0.2; exendin-4-Lys.sup.40(8-MPA)-NH.sub.2; exendin-4-Lys.sup.40(.epsilon.-AEEA-AEEA-MPA)-NH.sub.2; exendin-4-Lys.sup.40(8-AEEA-MPA)-NH.sub.2; exendin-4-Lys.sup.40(.epsilon.-MPA)-albumin; exendin-4-Lys.sup.40(8-AEEA-AEEA-MPA)-albumin; exendin-4-Lys.sup.40(.epsilon.-AEEA-MPA)-albumin; and the like. AEEA refers to [2-(2-amino)ethoxy)]ethoxy acetic acid. EDA refers to ethylenediamine. MPA refers to maleimidopropionic acid. The exendins and exendin analogs may optionally be amidated. Other exendins and exendin analogs useful in the methods described herein include those described in WO 98/05351; WO 99/07404; WO 99/25727; WO 99/25728; WO 99/40788; WO 00/41546; WO 00/41548; WO 00/73331; WO 01/51078; WO 03/099314; U.S. Pat. Nos. 6,956,026; 6,506,724; 6,703,359; 6,858,576; 6,872,700; 6,902,744; 7,157,555; 7,223,725; 7,220,721; U.S. Publication No. 2003/0036504; and U.S. Publication No. 2006/0094652, the disclosures of which are incorporated by reference herein in their entirety.

As used herein, “pharmaceutically acceptable carrier” includes any material which, when combined with the reactive agent, retains the reactive agent's biological activity, e.g., binding specificity and is non-reactive with the subject's immune system. Examples include, but are not limited to, any of the standard pharmaceutical carriers such as a phosphate buffered saline solution, water, emulsions such as oil/water emulsion, and various types of wetting agents. Other carriers may also include sterile solutions, tablets, including coated tablets and capsules. Typically, such carriers contain excipients, such as starch, milk, sugar, certain types of clay, gelatin, stearic acid or salts, thereof, magnesium or calcium stearate, talc, vegetable fats or oils, gums, glycols, or other known excipients. Such carriers may also include flavor and color additives or other ingredients. Compositions comprising such carriers are formulated by well-known conventional methods.

As used herein, “immunosuppressive agents” are defined as a composition having one or more types of molecules that prevent the occurrence of an immune response, or weaken a subject's immune system. Preferably, the agents reduce or prevent T cell proliferation. Some agents may inhibit T cell proliferation by inhibiting interaction of T cells with other antigen presenting cells (APCs). One example of APCs is B cells. Examples of agents that interfere with T cell interactions with APCs, and thereby inhibit T cell proliferation, include, but are not limited to, ligands for CD80 and/or CD86 antigens, ligands for CTLA4 antigen, and ligands for CD28 antigen. Examples of ligands for CD80 and/or CD86 antigens include, but are not limited to, soluble CTLA4, soluble CTLA4 mutant, soluble CD28, or monoclonal antibodies that recognize and bind CD80 and/or CD86 antigens, or fragments thereof. One preferred agent is L104EA29YIg. Ligands for CTLA4 or CD28 antigens include monoclonal antibodies that recognize and bind CTLA4 and/or CD28, or fragments thereof. Other ligands for CTLA4 or CD28 include soluble CD80 and/or CD86 molecules, such as CD80 and/or CD86Ig. Persons skilled in the art will readily understand that other agents or ligands can be used to inhibit the interaction of CD28 with CD80 and/or CD86.

Immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE.sup.R), etanercept, TNF.alpha. blockers, a biological agent that targets an inflammatory cytokine, and Non-Steroidal Anti-Inflammatory Drug (NSAIDs). NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol. In one embodiment, immunosuppressive agents are administered with myeloid derived suppressor cells to prevent ovarian failure.

In some embodiments of the invention, therapeutic microvesicles are administered together with a “tolerogenic adjuvant”, or an “ovarian failure treatment adjuvant”. In one embodiment tolerogenic adjuvants is low dose interleukin-2. term “low-dose IL-2” refers to the dosage range wherein immune suppressive T cells are preferentially enhanced relative to Tcons. In one embodiment, low-dose IL-2 refers to IL-2 doses that are less than or equal to 50% of the “high-dose IL-2” doses (e.g., 18 million IU per m.sup.2 per day to 20 million IU per m.sup.2 per day, or more) used for anti-cancer immunotherapy. The upper limit of “low-dose IL-2” can further be limited by treatment adverse events, such as fever, chills, asthenia, and fatigue. IL-2 is generally dosed according to an amount measured in international units (IU) administered in comparison to body surface area (BSA) per given time unit. BSA can be calculated by direct measurement or by any number of well-known methods (e.g., the Dubois & Dubois formula), such as those described in the Examples. Generally, IL-2 is administered according in terms of IU per m.sup.2 of BSA per day. Exemplary low-dose IL-2 doses according to the methods of the present invention include, in terms of 10.sup.6 IU/m.sup.2/day, any one of 0.3, 0.4, 0.5, 0.6, 0.7, 0.8, 0.9, 1.0, 1.1, 1.2, 1.3, 1.4, 1.5, 1.6, 1.7, 1.8, 1.9, 2,0, 2.1, 2.2, 2.3, 2.4, 2.5, 2.6, 2.7, 2.8, 2.9, and 3.0.times.10.sup.6 IU/m.sup.2/day, including any values in between and/or ranges in between. For example, an induction regimen dose can range between 0.3.times.10.sup.6 IU/m.sup.2/day and 3.0.times.10.sup.6 IU/m.sup.2/day with any value or range in between.

The term “continuous administration” refers to administration of IL-2 at regular intervals without any intermittent breaks in between. Thus, no interruptions in IL-2 occur. For example, the induction dose can be administered every day (e.g., once or more per day) during at least 1-14 consecutive days or any range in between (e.g., at least 4-7 consecutive days). As described herein, longer acting IL-2 agents and/or IL-2 agents administered by routes other than subcutaneous administration are contemplated. Intermittent intravenous administration of IL-2 described in the art results in short IL-2 half lives incompatible with increasing plasma IL-2 levels and increasing the immune suppressive T cells:Tcons ratio according to the present invention. However, once-daily subcutaneous IL-2 dosing, continuous IV infusion, long-acting subcutaneous IL-2 formulations, and the like are contemplated for achieving a persistent steady state IL-2 level.

As described above, IL-2 can be administered in a pharmaceutically acceptable formulation and by any suitable administration route, such as by subcutaneous, intravenous, intraperitoneal, oral, nasal, transdermal, or intramuscular administration. In one embodiment, the present invention provides pharmaceutically acceptable compositions which compose IL-2 at a therapeutically-effective amount, formulated together with one or more pharmaceutically acceptable carriers (additives) and/or diluents. The pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

In some embodiments of the invention, said tolerogenic adjuvants are agents that increase T regulatory cell activity and/or number. In one embodiment said tolerogenic adjuvant is rapamycin. In another embodiment, said tolerogenic adjuvant is anti-CD3 antibodies.

For the practice of the invention, it is important to utilize the proper type of anti-CD3 antibody. The natural role of CD3 is to transduce signals in T cells from the T cell receptor into the nucleus of the T cells, usually to activity T cells. In some situations, antibodies to CD3 cause activation of T cells, not suppression. For example, Hirsch et al. investigated the ability of low dose anti-CD3 to enhance an anti-tumor response directed against the malignant murine UV-induced skin tumor. Low dose anti-CD3 administration resulted in enhanced in vitro anti-tumor activity and prevented tumor outgrowth in approximately two-thirds of animals treated at the time of tumor inoculation. Furthermore, these animals displayed lasting tumor-specific immunity. Augmentation of various parameters of immunity was noted. These results suggested that anti-CD3 mAb can be utilized for the enhancement of anti-tumor responses in vivo and may have general application in the treatment of immunodeficiency. They also point to the care that needs to be exercised when manipulating the CD3 pathway, given that the pathway can be both activatory or inhibitory [91]. Activatory signals by crosslinking CD3 are also seen in the tumor infiltrating lymphocyte (TIL) culture systems. It is known that early in the life of the TIL bulk culture, cytotoxicity is non-major histocompatibility complex restricted. Under these culture conditions antitumor cytotoxicity was observed to decline with increasing age of the bulk culture. In addition, TIL became refractory to IL-2-induced expansion. In one study, scientists have used solid-phase anti-CD3 antibodies for TIL activation followed by culture in reduced concentrations of IL-2 to reactivate TIL previously grown in high concentrations of rIL-2. TIL refractory to IL-2 in terms of growth and antitumor cytotoxicity proved sensitive to anti-CD3 activation. The use of solid-phase anti-CD3 was also more effective than high concentrations of IL-2 in the expansion of TIL when used at the start of culture. Finally, TIL could be induced to secrete IL-2 following solid-phase activation with anti-CD3. These data suggest that human TIL are susceptible to activation by signals directed at the CD3 complex of the TIL cell surface [92].

An example of how different CD3 targeting antibodies can elicit different effects is seen in another study, which Davis et al. examined the IgM monoclonal antibody called 38.1, which was distinct from other anti-CD3 mAb, in that it was rapidly modulated from the cell surface in the absence of a secondary antibody. Although 38.1 induced an immediate increase in intracellular free calcium [Ca2+]i by highly purified T cells, it did not induce entry of the cells into the cell cycle in the absence of accessory cells (AC) or a protein kinase C-activating phorbol ester. Treated T cells were markedly inhibited in their capacity to respond to the T cell stimulating mitogen phytohemagluttanin. Inhibition of responsiveness could be overcome by culturing the cells with supplemental antigen presenting cells or the cytokine IL-2. These studies demonstrate that a state of T cell nonresponsiveness can be induced by modulating CD3 with an anti-CD3 mAb in the absence of co-stimulatory signals. A brief increase in [Ca2+]i resulting from mobilization of internal calcium stores appears to be sufficient to induce this state of T cell nonresponsiveness [93].

In some situations, anti-CD3 antibodies have been shown to program T cells towards antigen-specific tolerance. This is illustrated in one example in the work of Anasetti et al. who exposed PBMC to alloantigen for 3-8 d in the presence of anti-CD3 antibodies. They showed no response after restimulation with cells from the original donor but the PBMC remained capable of responding to third-party donors. Antigen-specific nonresponsiveness was induced by both nonmitogenic and mitogenic anti-CD3 antibodies but not by antibodies against CD2, CD4, CD5, CD8, CD18, or CD28. This suggested the unique ability of this protein to modulate programs in the T cells that are antigen specific. Nonresponsiveness induced by anti-CD3 antibody in mixed leukocyte culture was sustained for at least 34 d from initiation of the culture and 26 d after removal of the antibody. Anti-CD3 antibody also induced antigen-specific nonresponsiveness in cytotoxic T cell generation assays. Anti-CD3 antibody did not induce nonresponsiveness in previously primed cells [94].

The use of anti-CD3 antibodies for the practice of the invention requires that the antibodies not only do not result in activation of T cell proliferation and inflammatory cytokine secretion, but also that the T cells actually inhibit inflammation and promote regeneration.

In one embodiment of the invention, anti-CD3 antibody is given 14 days before administration of mesenchymal stem cells In one specific embodiment, said 14-day course of the anti-CD3 monoclonal antibody utilizes the antibody hOKT3γ1(Ala-Ala) administered intravenously (1.42 μg per kilogram of body weight on day 1; 5.67 μg per kilogram on day 2; 11.3 μg per kilogram on day 3; 22.6 μg per kilogram on day 4; and 45.4 μg per kilogram on days 5 through 14); these doses were based on those previously used for treatment of transplant rejection [95] which is incorporated by reference. Other types of anti-CD3 molecules and dosing regimens may be used in the context of ARDS therapeutics, said doses may be chosen from examples of utility of anti-CD3 from the literature, as described in the following papers and incorporated by reference: prevention of kidney [96-104], liver [105-107], pancreas [108-110], lung [111], and heart [112-116] transplant rejection; prevention of graft versus host disease [117], multiple sclerosis [118], type 1 diabetes [119],

The use of monoclonal antibodies for the practice of the invention must be tempered by the caution that in some cases cytokine storm may be initiated by antibody administration [120, 121]. In some cases this is concentration dependent [122]. Treatment for this can be accomplished by steroid administration or anti-IL6 antibody [123-127]. Additionally, the patent discloses that anti-IL-6 may be administer inside the ovary in part for suppression of ongoing inflammatory response.

In some embodiments of the invention administration of PGE1 and/or various natural anti-inflammatory compounds are provided to decrease TNF-alpha production as a result of anti-CD3 administration, such as described in this paper and incorporated by reference [128]. In further embodiments of the invention, administration of anti-CD3 may be performed together with endothelial protectants and/or anti-coagulants in order to reduce clotting associated with CD3 modulating agents [129]. In some embodiments anti-CD3 antibodies may be used in combination with tolerogenic cytokines such as interleukin-10 in order to enhance number of angiogenesis supporting T cells. The safety of anti-CD3 and IL-10 administration has previously been demonstrated in a clinical trial [130].

In the current invention decreased TNF-alpha activity is correlated with enhancement of ovarian regenerative activity. Furthermore, other inhibitors of TNF-alpha may be administered [131, 132]. Administration of mesenchymal stem cell exosomes may be performed into the ovary directly as a means of treating ovarian failure. A “mesenchymal stem cell (MSC)” is a progenitor cell having the capacity to differentiate into neuronal cells, adipocytes, chondrocytes, osteoblasts, myocytes, cardiac tissue, and other endothelial or epithelial cells. These cells may be defined phenotypically by gene or protein expression. These cells have been characterized to express (and thus be positive for) one or more of CD13, CD29, CD44, CD49a, b, c, e, f, CD51, CD54, CD58, CD71, CD73, CD90, CD102, CD105, CD106, CDw119, CD120a, CD120b, CD123, CD124, CD126, CD127, CD140a, CD166, P75, TGF-bIR, TGF-bIIR, HLA-A, B, C, SSEA-3, SSEA-4, D7 and PD-L1. These cells have also been characterized as not expressing (and thus being negative for) CD3, CD5, CD6, CD9, CD10, CD11a, CD14, CD15, CD18, CD21, CD25, CD31, CD34, CD36, CD38, CD45, CD49d, CD50, CD62E, L, S, CD80, CD86, CD95, CD117, CD133, SSEA-1, and ABO. Thus, MSCs may be characterized phenotypically and/or functionally according to their differentiation potential.

Commercially available media may be used for the growth, culture and maintenance of MSCs. Such media include but are not limited to Dulbecco's modified Eagle's medium (DMEM). Components in such media that are useful for the growth, culture and maintenance of MSCs, fibroblasts, and macrophages include but are not limited to amino acids, vitamins, a carbon source (natural and non-natural), salts, sugars, plant derived hydrolysates, sodium pyruvate, surfactants, ammonia, lipids, hormones or growth factors, buffers, non-natural amino acids, sugar precursors, indicators, nucleosides and/or nucleotides, butyrate or organics, DMSO, animal derived products, gene inducers, non-natural sugars, regulators of intracellular pH, betaine or osmoprotectant, trace elements, minerals, non-natural vitamins. Additional components that can be used to supplement a commercially available tissue culture medium include, for example, animal serum (e.g., fetal bovine serum (FBS), fetal calf serum (FCS), horse serum (HS)), antibiotics (e.g., including but not limited to, penicillin, streptomycin, neomycin sulfate, amphotericin B, blasticidin, chloramphenicol, amoxicillin, bacitracin, bleomycin, cephalosporin, chlortetracycline, zeocin, and puromycin), and glutamine (e.g., L-glutamine). Mesenchymal stem cell survival and growth also depends on the maintenance of an appropriate aerobic environment, pH, and temperature. MSCs can be maintained using methods known in the art, e.g., as described in Pittenger et al., Science, 284:143-147 (1999), incorporated herein by reference.

In some embodiments, the MSC exosomes used to treat the ovaries are isolated. As used herein, an “isolated exosome” is an exosome that is physically separated from its natural environment. An isolated exosome may be physically separated, in whole or in part, from tissue or cells with which it naturally exists (e.g., MSCs). In some embodiments, the isolated MSC exosomes are isolated from the culturing media of MSCs from human bone marrow, umbilical cord Wharton's Jelly, or adipose tissue. Such culturing media is termed “MSC-conditioned media” herein. In some embodiments, isolated exosomes may be free of cells such as MSCs, or it may be free or substantially free of conditioned media, or it may be free of any biological contaminants such as proteins. Typically, the isolated exosomes are provided at a higher concentration than exosomes present in un-manipulated conditioned media. In some embodiments, exosomes are collected from mesenchymal stem cells that have been stimulated.

In one embodiment, activated, or “stimulated” mesenchymal stem cells are treated with conditions such as “hypoxia”, exposed to inflammatory stimuli such as cytokines and/or toll like receptor agonists. In some embodiments, the isolated MSC exosome described herein comprises one or more (e.g., 1, 2, 3, 4, 5, or more) known exosome markers. In some embodiments, the known exosome markers are selected from the group consisting of: FLOT1 (Flotillin-1, Uniprot ID: 075955), CD9 (CD9 antigen, Uniprot ID: P21926), and CD63 (CD63 antigen, Uniprot ID: P08962). In some embodiments, the isolated MSC exosome is substantially free of contaminants (e.g., protein contaminants). The isolated MSC exosome is “substantially free of contaminants” when the preparation of the isolated MSC exosome contains fewer than 20%, 15%, 10%, 5%, 2%, 1%, or less than 1%, of any other substances (e.g., proteins). In some embodiments, the isolated MSC is “substantially free of contaminants” when the preparation of the isolated MSC exosome is at least 80%, at least 85%, at least 90%, at least 95%, at least 98%, at least 99%, at least 99.9% pure, with respect to contaminants (e.g., proteins). “Protein contaminants” refer to proteins that are not associated with the isolated exosome and do not contribute to the biological activity of the exosome. The protein contaminants are also referred to herein as “non-exosomal protein contaminants.” In some embodiments, the isolated MSC exosome used in accordance with the present disclosure has a diameter of about 30-150 nm. For example, the isolated MSC exosome may have a diameter of 30-150 nm, 30-140 nm, 30-130 nm, 30-120 nm, 30-110 nm, 30-100 nm, 30-90 nm, 30-80 nm, 30-70 nm, 30-60 nm, 30-50 nm, 30-40 nm, 40-150 nm, 40-140 nm, 40-130 nm, 40-120 nm, 40-110 nm, 40-100 nm, 40-90 nm, 40-80 nm, 40-70 nm, 40-60 nm, 40-50 nm, 50-150 nm, 50-140 nm, 50-130 nm, 50-120 nm, 50-110 nm, 50-100 nm, 50-90 nm, 50-80 nm, 50-70 nm, 50-60 nm, 60-150 nm, 60-140 nm, 60-130 nm, 60-120 nm, 60-110 nm, 60-100 nm, 60-90 nm, 60-80 nm, 60-70 nm, 70-150 nm, 70-140 nm, 70-130 nm, 70-120 nm, 70-110 nm, 70-100 nm, 70-90 nm, 70-80 nm, 80-150 nm, 80-140 nm, 80-130 nm, 80-120 nm, 80-110 nm, 80-100 nm, 80-90 nm, 90-150 nm, 90-140 nm, 90-130 nm, 90-120 nm, 90-110 nm, 90-100 nm, 100-150 nm, 100-140 nm, 100-130 nm, 100-120 nm, 100-110 nm, 110-150 nm, 110-140 nm, 110-130 nm, 110-120 nm, 120-150 nm, 120-140 nm, 120-130 nm, 130-150 nm, 130-140 nm, or 140-150 nm. In some embodiments, the isolated MSC exosome may have a diameter of about 50 nm, 60 nm, 70 nm, 80 nm, 90 nm, 100 nm, 110 nm, 120 nm, 130 nm, 140 nm, or 150 nm. In some embodiments, the isolated MSC exosomes exhibit a biconcave morphology. As described herein, the isolated MSC exosomes can be used to treat the monocytes to modulate the monocyte phenotype (e.g., both in vitro and in vivo such as in the bone marrow). “Treat a monocyte with an isolated MSC exosome” means contacting the monocyte with a MSC exosome (e.g., for a period of time). In some embodiments, the treating (i.e., contacting) is carried out in vitro. For example, monocytes may be cultured in vitro and isolated MSC exosomes may be added to the culture such that the monocytes contact the isolated MSC exosomes. In some embodiments, the treating (i.e., contacting) is carried out ex vivo. For example, monocytes may be isolated from the bone marrow of a subject and isolated MSC exosomes may be added to the monocytes such that the monocytes contact the isolated MSC exosomes. In some embodiments, the treating (i.e., contacting) is carried out in vivo. For example, the isolated MSC exosomes may be administered to a subject (e.g., via intravenous injection), reach the one marrow, and contact the monocytes in the bone marrow. In some embodiments, the monocyte is treated (i.e., contacted) with the MSC exosome for at least 1 hour (e.g., at least 1, at least 2, at least 3, at least 4, at least 5, at least 6, a least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 hours, or longer). In some embodiments, the monocyte is treated (i.e., contacted) with the MSC exosome for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 hours, or longer. In some embodiments, the monocyte has been polarized to a pro-inflammatory state as a result of environmentally or developmentally-precipitated injury, and its polarity is modulated to a regulatory phenotype upon contact with the isolated MSC exosome. In some embodiments, the monocyte is a pro-inflammatory monocyte prior to being treated (i.e., contacted) with the isolated MSC exosome, and is a regulatory monocyte after being treated (i.e., contacted) with the isolated MSC exosome. In some embodiments, a mixture of pro-inflammatory monocytes and regulatory monocytes are contacted with isolated MSC exosomes and the treating results in a higher ratio (e.g., at least 10% higher) of regulatory monocytes in the mixture, being treated with isolated MSC exosomes. For example, the ratio of regulatory monocytes may be at least 10%, at least 20%, at least 30%, at least 40%, at least 50%, at least 60%, at least 70%, at least 80%, at least 90%, at least 100%, at least 2-fold, at least 5-fold, at least 10-fold, at least 100-fold, or higher after being treated with MSC exosomes, compared to before being treated with isolated MSC exosomes. In some embodiments, the ratio of regulatory monocytes is 10%, 20%, 30%, 40%, 50%, 60%, 70%, 80%, 90%, 100%, 2-fold, 5-fold, 10-fold, 100-fold, or higher after being treated with MSC exosomes, compared to before being treated with isolated MSC exosomes. Further provided herein are uses of the monocytes treated with isolated MSC exosomes for treating a disease (e.g., a fibrotic disease such as pulmonary fibrosis or an autoimmune disease). In some embodiments, the monocytes treated with isolated MSC exosomes are used in the manufacturing of a medicament for treating a disease (e.g., a fibrotic disease or an autoimmune disease). Compositions comprising monocytes treated with isolated MSC exosomes are also provided. In some embodiments, the monocytes treated with isolated MSC exosomes are formulated in a composition for the treatment of ovarian failure. In some embodiments, the composition comprising monocytes treated with isolated MSC exosomes further comprises a second agent.

In some embodiments ovaries are treated with monocyte exosomes and/or agents which suppress fibrosis. Exemplary compounds that may be used to treat fibrotic diseases include, without limitation: nintedanib (a tyrosine kinase inhibitor), pirfenidone, an anti-fibrotic agent, and/or an anti-inflammatory agent. In some embodiments, for pulmonary fibrosis, other types of therapies, e.g., oxygen supplement, may be used in conjunction with the therapeutic agents described herein.

In some embodiments of the invention, enhancement of ovarian regenerative activity is provided by administration of oral modulators of CD3. Oral administration of OKT3 has been previously performed in a clinical trial and results are incorporated by reference [133, 134].

In one embodiment, MSC exosomes, or particles may be produced by culturing mesenchymal stem cells in a medium to condition it. The mesenchymal stem cells may comprise human umbilical tissue derived cells which possess markers selected from a group comprising of CD90, CD73 and CD105. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or b-mercaptoethanol, or any combination thereof. The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a>1000 kDa membrane. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6.times.40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The rh of particles in this peak is about 45-55 nm. Such fractions comprise mesenchymal stem cell particles such as exosomes.

Culture conditioned media may be concentrated by filtering/desalting means known in the art. In one embodiment Amicon filters, or substantially equivalent means, with specific molecular weight cut-offs are utilized, said cut-offs may select for molecular weights higher than 1 kDa to 50 kDa.

The cell culture supernatant may alternatively be concentrated using means known in the art such as solid phase extraction using C18 cartridges (Mini-Spe-ed C18-14%, S.P.E. Limited, Concord ON). Said cartridges are prepared by washing with methanol followed by deionized-distilled water. Up to 100 ml of stem cell or progenitor cell supernatant may be passed through each of these specific cartridges before elution, it is understood of one of skill in the art that larger cartridges may be used. After washing the cartridges material adsorbed is eluted with 3 ml methanol, evaporated under a stream of nitrogen, redissolved in a small volume of methanol, and stored at 4.degree. C.

Before testing the eluate for activity in vitro, the methanol is evaporated under nitrogen and replaced by culture medium. Said C18 cartridges are used to adsorb small hydrophobic molecules from the stem or progenitor cell culture supernatant, and allows for the elimination of salts and other polar contaminants. It may, however be desired to use other adsorption means in order to purify certain compounds from said fibroblast cell supernatant. Said fibroblast concentrated supernatant may be assessed directly for biological activities useful for the practice of this invention, or may be further purified. In one embodiment, said supernatant of fibroblast culture is assessed for ability to stimulate proteoglycan synthesis using an in vitro bioassay. Said in vitro bioassay allows for quantification and knowledge of which molecular weight fraction of supernatant possesses biological activity. Bioassays for testing ability to stimulate proteoglycan synthesis are known in the art. Production of various proteoglycans can be assessed by analysis of protein content using techniques including mass spectrometry, column chromatography, immune based assays such as enzyme linked immunosorbent assay (ELISA), immunohistochemistry, and flow cytometry.

Further purification may be performed using, for example, gel filtration using a Bio-Gel P-2 column with a nominal exclusion limit of 1800 Da (Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5.times.54 cm glass column and equilibrated with 3 column volumes of the same buffer. Amniotic fluid stem cell supernatant concentrates extracted by C18 cartridge may be dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically.

In one embodiment of the invention therapeutic exosomes are administered together with “tolerogenic adjuvants”. Adjuvants of use may include exenatide, alpha-1-trypsin, nicotinamide [135], aminoguanidine [136], or GM-CSF.

In some embodiments the invention teaches the administration of therapeutic microvesicles as a means of inhibiting fibrosis associated with ovarian degeneration. In some embodiments therapeutic microvesicles such as exosomes are administered in situations of mixed hematopoietic chimerism to increase chimerism. In some embodiments microvesicles a donor derived.

In some embodiments of the invention administration of therapeutic microvesicles is performed together with cellular therapy for diabetes. In one embodiment cellular therapy comprises administration of islet cells. Protocols for islet cell transplantation are known in the literature and incorporated by reference. In other embodiments regenerative cells are administered together with islets or as a monotherapy [219]. For example, in one paper a nonrandomized, open-label, parallel-armed prospective study was described. MSCs were isolated from umbilical cord (UC) of healthy donors. Fifty-three participants including 33 adult-onset (>18 years) and 20 juvenile-onset T1D were enrolled. Twenty-seven subjects (MSC-treated group) received an initial systemic infusion of allogeneic UC-MSCs, followed by a repeat course at 3 months, whereas the control group (n=26) only received standard care based on intensive insulin therapy. Data at 1-year follow-up was reported in this study. The primary endpoint was clinical remission defined as a 10% increase from baseline in the level of fasting and/or postprandial C-peptide. The secondary endpoints included side effects, serum levels of HbA1c, changes in fasting and postprandial C-peptide, and daily insulin doses. After 1-year follow-up, 40.7% subjects in MSC-treated group achieved the primary endpoint, significantly higher than that in the control arm. Three subjects in MSC-treated group, in contrast to none in control group, achieved insulin independence and maintained insulin free for 3 to 12 months. Among the adult-onset T1D, the percent change of postprandial C-peptide was significantly increased in MSC-treated group than in the control group. However, changes in fasting or postprandial C-peptide were not significantly different between groups among the juvenile-onset T1D. Multivariable logistic regression assay indicated that lower fasting C-peptide and higher dose of UC-MSC correlated with achievement of clinical remission after transplantation. No severe side effects were observed. It was concluded that one repeated intravenous dose of allogeneic UC-MSCs is safe in people with recent-onset T1D and may result in better islet R cell preservation during the first year after diagnosis compared to standard treatment alone [220].

The invention includes pharmaceutical compositions for use in the treatment of ovarian failure comprising pharmaceutically effective amounts of immune modulatory microvesicles. In some embodiments said microvesicles are administered together with soluble CTLA4 mutant molecules. In certain embodiments, the immune system diseases are mediated by CD28- and/or CTLA4-positive cell interactions with CD80 and/or CD86 positive cells. The soluble CTLA4 molecules are preferably soluble CTLA4 molecules with wildtype sequence and/or soluble CTLA4 molecules having one or more mutations in the extracellular domain of CTLA4. The pharmaceutical composition can include soluble CTLA4 or CTLA4 mutant protein molecules and/or nucleic acid molecules, and/or vectors encoding the molecules. In a preferred embodiment, the soluble CTLA4 mutant molecule has the amino acid sequence of the extracellular domain of CTLA4 as shown in either FIG. 3 (L104EA29Y). Even more preferably, the soluble CTLA4 mutant molecule is L104EA29YIg as disclosed herein shown in FIG. 3. The compositions may additionally include other therapeutic agents, including, but not limited to, immunosuppressive agents, NSAIDs, corticosteroids, glucococoticoids, drugs, toxins, enzymes, antibodies, or conjugates. An embodiment of the pharmaceutical composition comprises an effective amount of a therapeutic microvesicles alone or in combination with an effective amount of at least one other therapeutic agent, including an immunosuppressive agent, or NSAID. Effective amounts of the therapeutic microvesicle in the pharmaceutical composition can range about 0.1 pg to 100 mg/kg weight of the subject. In another embodiment, the effective amount can be an amount about 0.5 to 5 mg/kg weight of a subject, about 5 to 10 mg/kg weight of a subject, about 10 to 15 mg/kg weight of a subject, about 15 to 20 mg/kg weight of a subject, about 20 to 25 mg/kg weight of a subject, about 25 to 30 mg/kg weight of a subject, about 30 to 35 mg/kg weight of a subject, about 35 to 40 mg/kg weight of a subject, about 40 to 45 mg/kg of a subject, about 45 to 50 mg/kg weight of a subject, about 50 to 55 mg/kg weight of a subject, about 55 to 60 mg/kg weight of a subject, about 60 to 65 mg/kg weight of a subject, about 65 to 70 mg/kg weight of a subject, about 70 to 75 mg/kg weight of a subject, about 75 to 80 mg/kg weight of a subject, about 80 to 85 mg/kg weight of a subject, about 85 to 90 mg/kg weight of a subject, about 90 to 95 mg/kg weight of a subject, or about 95 to 100 mg/kg weight of a subject. In an embodiment, the effective amount is 2 mg/kg weight of a subject. In another embodiment, the effective amount is 10 mg/kg weight of a subject. In an embodiment, the effective amount of a soluble CTLA4 molecule is 2 mg/kg weight of a subject. In an embodiment, the effective amount of a soluble CTLA4 molecule is 10 mg/kg weight of a subject. The amount of an immunosuppressive agent administered to a subject varies depending on several factors including the efficacy of the drug on a specific subject and the toxicity (i.e. the tolerability) of a drug to a specific subject. Methotrexate is commonly administered in an amount about 0.1 to 40 mg per week with a common dosage ranging about 5 to 30 mg per week. Methotrexate may be administered to a subject in various increments: about 0.1 to 5 mg/week, about 5 to 10 mg/week, about 10 to 15 mg/week, about 15 to 20 mg/week, about 20 to 25 mg/week, about 25 to 30 mg/week, about 30 to 35 mg/week, or about 35 to 40 mg/week. In one embodiment, an effective amount of an immunosuppressive agent, including methotrexate, is an amount about 10 to 30 mg/week. Effective amounts of methotrexate range about 0.1 to 40 mg/week. In one embodiment, the effective amount is ranges about 0.1 to 5 mg/week, about 5 to 10 mg/week, about 10 to 15 mg/week, about 15 to 20 mg/week, about 20 to 25 mg/week, about 25 to 30 mg/week, about 30 to 35 mg/week, or about 35 to 40 mg/week. In one embodiment, methotrexate is administered in an amount ranging about 10 to 30 mg/week. Cyclophosphamide, an alkylating agent, may be administered in dosages ranging about 1 to 10 mg/kg body weight per day. Cyclosporine (e.g. NEORAL) also known as Cyclosporin A, is commonly administered in dosages ranging from about 1 to 10 mg/kg body weight per day. Dosages ranging about 2.5 to 4 mg per body weight per day are commonly used. Chloroquine or hydroxychloroquine (e.g. PLAQUENIL), is commonly administered in dosages ranging about 100 to 1000 mg daily. Preferred dosages range about 200-600 mg administered daily. Sulfasalazine (e.g., AZULFIDINE EN-tabs) is commonly administered in amounts ranging about 50 to 5000 mg per day, with a common dosage of about 2000 to 3000 mg per day for adults. Dosages for children are commonly about 5 to 100 mg/kg of body weight, up to 2 grams per day. Gold salts are formulated for two types of administration: injection or oral. Injectable gold salts are commonly prescribed in dosages about 5 to 100 mg doses every two to four weeks. Orally administered gold salts are commonly prescribed in doses ranging about 1 to 10 mg per day. D-penicillamine or penicillamine (CUPRIMINE) is commonly administered in dosages about 50 to 2000 mg per day, with preferred dosages about 125 mg per day up to 1500 mg per day. Azathioprine is commonly administered in dosages of about 10 to 250 mg per day. Preferred dosages range about 25 to 200 mg per day. Anakinra (e.g. KINERET) is an interleukin-1 receptor antagonist. A common dosage range for anakinra is about 10 to 250 mg per day, with a recommended dosage of about 100 mg per day. Infliximab (REMICADE) is a chimeric monoclonal antibody that binds to tumor necrosis factor alpha (TNF.alpha.). Infliximab is commonly administered in dosages about 1 to 20 mg/kg body weight every four to eight weeks. Dosages of about 3 to 10 mg/kg body weight may be administered every four to eight weeks depending on the subject. Etanercept (e.g. ENBREL) is a dimeric fusion protein that binds the tumor necrosis factor (TNF) and blocks its interactions with TNF receptors. Commonly administered dosages of etanercept are about 10 to 100 mg per week for adults with a preferred dosage of about 50 mg per week. Dosages for juvenile subjects range about 0.1 to 50 mg/kg body weight per week with a maximum of about 50 mg per week.

Enhancement of therapeutic activity of exosomes in treatment of ovarian failure may be induced by administration of Leflunomide (ARAVA), which is commonly administered at dosages about 1 and 100 mg per day. A common daily dosage is about 10 to 20 mg per day. The pharmaceutical compositions also preferably include suitable carriers and adjuvants which include any material which when combined with the molecule of the invention (e.g., a soluble CTLA4 mutant molecule, e.g., L104EA29YIg) retains the molecule's activity and is non-reactive with the subject's immune system. Examples of suitable carriers and adjuvants include, but are not limited to, human serum albumin; ion exchangers; alumina; lecithin; buffer substances, such as phosphates; glycine; sorbic acid; potassium sorbate; and salts or electrolytes, such as protamine sulfate. Other examples include any of the standard pharmaceutical carriers such as a phosphate buffered saline solution; water; emulsions, such as oil/water emulsion; and various types of wetting agents. Other carriers may also include sterile solutions; tablets, including coated tablets and capsules. Typically, such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well-known conventional methods. Such compositions may also be formulated within various lipid compositions, such as, for example, liposomes as well as in various polymeric compositions, such as polymer microspheres.

The pharmaceutical compositions of the invention can be administered using conventional modes of administration including, but not limited to, intravenous (i.v.) administration, intraperitoneal (i.p.) administration, intramuscular (i.m.) administration, subcutaneous administration, oral administration, administration as a suppository, or as a topical contact, or the implantation of a slow-release device such as a miniosmotic pump, to the subject.

The pharmaceutical compositions of the invention may be in a variety of dosage forms, which include, but are not limited to, liquid solutions or suspensions, tablets, pills, powders, suppositories, polymeric microcapsules or microvesicles, liposomes, and injectable or infusible solutions. The preferred form depends upon the mode of administration and the therapeutic application.

The most effective mode of administration and dosage regimen for the compositions of this invention depends upon the severity and course of the disease, the patient's health and response to treatment and the judgment of the treating physician. Accordingly, the dosages of the compositions should be titrated to the individual patient.

The invention provides novel compositions of matter for treatment of ovarian failure with exosomes, stem cell exosomes, and exosomes generated by monocytes that have been cultured with stem cells. This includes microvesicles derived from various cell types, methods of manufacture, and therapeutic uses. Provided are means of exosomes derived from myeloid cells reprogrammed by regenerative cells in which said myeloid cells possess regenerative, immune modulatory, anti-inflammatory, and angiogenic/neurogenic activity after culture or exposure to conditioned media from regenerative cells. One type of regenerative cell useful for the current invention is derived from umbilical cord tissue such as Wharton's Jelly. In some embodiments manipulation of stem cell “potency” is disclosed through hypoxic manipulation, growth on non-xenogeneic conditions, as well as addition of epigenetic modulators. The regenerative cells of the invention may be cultured under hypoxia, in one embodiment, cultured in order to induce and/or augment expression of chemokine receptors. One such receptor is CXCR-4. The population of cells, including population of umbilical cord mesenchymal cells, may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition, or alternatively, <1%, <2%, <3%, <4%, <5%, <6%, <7%, <8%, <9%, or <10% of the population of cells may express CD14 and/or CD45. The umbilical cord cells of the invention may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof. In some embodiments said placental cells of the invention are admixed with endothelial cells. Said endothelial cells may express one or more markers selected from the group consisting of: a) extracellular vimentin; b) CD133; c) c-kit; d) VEGF receptor; e) activated protein C receptor; and f) a combination thereof. In some embodiments, the population of endothelial cells comprises endothelial progenitor cells. The population of cells may be allogeneic, autologous, or xenogenic to an individual, including an individual being administered the population of cells. In some embodiments, the population of cells are matched by mixed lymphocyte reaction matching.

The generation of dendritic cells, which have been conditioned by exposure to culture supernatant of regenerative cells such as mesenchymal stem cells is disclosed in the current invention. In one embodiment the invention teaches that dendritic cells can be utilized after reprogramming but in some situations before reprogramming they may be pulsed with antigens. Dendritic cell technologies are disclosed in the following papers and incorporated by reference. [221-345]. In some embodiments, the population of cells is derived from tissue selected from the group consisting of the placental body, placenta, umbilical cord tissue, peripheral blood, hair follicle, cord blood, Wharton's Jelly, menstrual blood, endometrium, skin, omentum, amniotic fluid, and a combination thereof. In some embodiments, the population of cells, the population of umbilical mesenchymal stem cells, or the population of endothelial cells comprises human umbilical cord derived adherent cells. The human umbilical cord derived adherent cells may express a cytokines selected from the group consisting of) FGF-1; b) FGF-2; c) HGF; d) interleukin-1 receptor antagonist; and e) a combination thereof. In some embodiments, the population of cells, the population of umbilical cord cells express arginase, indoleamine 2,3 deoxygenase, interleukin-10, and/or interleukin 35. In some embodiments, the population of cells, the population of umbilical cord cells, or the population of endothelial cells express hTERT and Oct-4 but does not express a STRO-1 marker.

In some embodiments, the population of cells, the population of umbilical cord cells has an ability to undergo cell division in less than 36 hours in a growth medium. In some embodiments, the population of cells, the population of umbilical cord cells has an ability to proliferate at a rate of 0.9-1.2 doublings per 36 hours in growth media. In some embodiments, the population of cells, the population of umbilical cord cells has an ability to proliferate at a rate of 0.9, 1.0, 1.1, or 1.2 doublings per 36 hours in growth media. The population of cells, population of umbilical cord cells may produce exosomes capable of inducing more than 50% proliferation when the exosomes are cultured with human umbilical cord endothelial cells. The induction of proliferation may occur when the exosomes are cultured with the human umbilical cord endothelial cells at a concentration of 10, 20, 30, 40, 50, 60, 70, 80, 90, 100, 110, 120, 130, 140, 150, 160, 170, 180, 190, 200, or more exosomes per cell.

In some embodiments, a population of cells, including a population of umbilical cells alone, are administered to an individual, including an individual having and acute or chronic pathology, wherein the population of cells may be administered via any suitable route, including as non-limiting examples, intramuscularly and/or intravenously.

In some embodiments, a population of umbilical cord cells is optionally obtained, the population is then optionally contacted via culturing with a population of progenitor for T regulatory cells, wherein the culturing conditions allow for the generation of T regulatory cells, then the generated T regulatory cells are administered to an individual.

In another embodiment of the invention, biologically useful immune cells are generated after culture with regenerative cells, and/or stem cells are disclosed, of the mesenchymal or related lineages, which are therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has led to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory. During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be affected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching, or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may be increase proliferation, in other embodiments, it may be enhanced chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells. In order to induce therapeutic reprogramming of cells, in some cases, as disclosed herein, of wharton's jelly originating cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation. In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in G0 phase) and therefore can have a methylation status than conveys greater plasticity for reprogramming. Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell.

In some embodiments, host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need or repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1.times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10.sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

In some embodiments of the invention, administration of cells of the invention is performed for suppression of an inflammatory and/or autoimmune disease for treatment of ovarian failure. In these situations, it may be necessary to utilize an immune suppressive/or therapeutic adjuvant. Immune suppressants are known in the art and can be selected from a group comprising of: cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-α inhibitors, TNF-α sequestration agents, and methotrexate. More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

1. A method of preventing or treating ovarian failure comprising the steps of: a) identifying a patient suffering from ovarian failure or at risk of ovarian failure; b) withdrawing from said patient a population of myeloid lineage cells; c) contacting said myeloid lineage cells with a mesenchymal stem cell population and/or products generated from said mesenchymal stem cell population; d) pulsing said myeloid cell population with one or more antigens associated with ovarian failure; e) extracting microvesicles from said myeloid cell population; and f) administering said microvesicles from said myeloid cell population into the patient.

2. The method of claim 1, wherein said risk of ovarian failure is quantified by one or more selected from a group consisting of: a) increase production of interferon gamma from T cells responding to a ovarian antigen as compared to T cells from an age-matched subject; b) decreased production of interleukin-4 from T cells responding to a ovarian antigen as compared to T cells from an age-matched subject; c) increased antibodies to a ovarian antigen as compared to T cells from an age-matched subject; d) increased fibrosis in the ovarian follicle; and e) decreased production of estrogen.

3. The method of claim 2, wherein said T cells are selected from the group consisting of: a) CD3 T cells; b) CD4 T cells; c) CD8 T cells; d) Th1 T cells; e) Th2; f) Th3 T cells; g) Th9 T cells; h) Th17 T cells and i) Th22 T cells.

4. The method of claim 2, wherein said antibody is a complement fixing antibody.

5. The method of claim 2, wherein said antibody possesses the isotype IgG2b.

6. The method of claim 1, wherein said mesenchymal stem cells are derived from tissues.

7. The method of claim 6, wherein said tissue derived mesenchymal stem cells are selected from the group consisting of: a) bone marrow; b) perivascular tissue; c) adipose tissue; d) placental tissue; e) amniotic membrane; f) omentum; g) tooth; h) umbilical cord tissue-perinatal; i) fallopian tube tissue; j) hepatic tissue; k) renal tissue; 1) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

8. The method of claim 7, wherein said tissue derived mesenchymal stem cells are isolated from tissues containing cells selected from the group consisting of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, and salivary gland mucous cells.

9. The method of claim 1, wherein said mesenchymal stem cells are plastic adherent.

10. The method of claim 1, wherein said mesenchymal stem cells express a marker selected from the group consisting of: a) CD73; b) CD90; and c) CD105.

11. The method of claim 1, wherein said mesenchymal stem cells are derived from perinatal tissue and lack expression of a marker selected from the group consisting of: a) CD14; b) CD45; and c) CD34.

12. The method of claim 11, wherein said mesenchymal stem cells from perinatal tissue express markers selected from the group consisting of: a) oxidized low density lipoprotein receptor 1; b) chemokine receptor ligand 3; and c) granulocyte chemotactic protein.

13. The method of claim 11, wherein said mesenchymal stem cells from perinatal tissue do not express markers selected from the group consisting of: a) CD117; b) CD31; c) CD34; and CD45.

14. The method of claim 11, wherein said mesenchymal stem cells from perinatal tissue express, relative to a human fibroblast, increased levels of interleukin 8 and reticulon 1.

15. The method of claim 14, wherein said perinatal tissue-derived cells are positive for alkaline phosphatase staining.

16. The method of claim 14, wherein said perinatal tissue-derived cell secretes factors selected from the group consisting of: a) MCP-1; b) MIP1beta; c) IL-6; d) IL-8; e) GCP-2; f) HGF; g) KGF; h) FGF; i) HB-EGF; j) BDNF; k) TPO; 1) RANTES; and m) TIMP1.

17. The method of claim 14, wherein said perinatal tissue derived cells express markers selected from the group consisting of: a) TRA1-60; b) TRA1-81; c) SSEA3; d) SSEA4; and e) NANOG.

18. The method of claim 1, wherein said microvesicles are exosomes.

19. The method of claim 1, wherein said microvescicles are apoptotic bodies.

20. The method of claim 1, wherein said progenitor cells of the myeloid and granulocytic lineages are administered directly into the ovary.
